(19) United States
(12) Patent Application Publication
(10) Pub. No.: US 2021/0003495 A1
(43) Pub. Date: Jan. 7, 2021

(54) Method and apparatus to collect, fractionate, and classify airborne particles within specified volumetric regions

(71) Applicant: Philip Joseph Wyatt, Santa Barbara, CA (US)

(72) Inventor: Philip Joseph Wyatt, Santa Barbara, CA (US)

(21) Appl. No.: 16/501,931

(22) Filed: Jul. 3, 2019

(51) Int. Cl. G01N 15/14; C12Q 1/04; G01N 1/18

(52) U.S. Cl. CPC G01N 15/1456; C12Q 1/04; G01N 1/18; G01N 2015/1006; G01N 2015/1493; G01N 2015/1497; G01N 2015/1481; G01N 2015/149

(57) ABSTRACT

A searching procedure based on the detection and separation by specific multiangle scattered light properties of a small set of particles sought from a sample containing far greater concentrations of irrelevant particles is presented whereby all the particulate content of a volumetric fraction of the air of a targeted facility is captured and the particles of potential importance are separated therefrom. The unique size ranges, multiangle light scattering characteristics, and physical properties associated with specific particulates sought (such as bacteria or spores from spore-forming bacteria or asbestos particles, for example), once extracted from the total populations collected, are then processed for further study/classification/actions, as required. For the case of airborne bacteria, such processes could be expected to reduce the incidence of hospital acquired infections, by providing an early warning of their presence and capturing specific exemplars thereof for future analyses. Similarly, the early detection of a bioterrorist attack in progress or the presence of dangerous airborne contaminants, such as asbestos fibers, will have long serving benefits.

### BACKGROUND

The detection, capture, and identification of certain classes of airborne particles, whose presence within a variety of local environments is considered potentially dangerous, represent tasks of significant importance. Among such particles, bacteria in hospital wards and patient facilities are considered of great consequence as such bacteria are often the source of hospital acquired infections (HAI). Unfortunately, the concentration of bacteria capable of causing a HAI is generally far below levels amenable to early detection. Early detection of other hazardous particles, such as asbestos created during various demolition and remodeling activities, are further examples of the airborne contaminants whose concentrations may be too low for real time detection by current processes.

Following the anthrax mailings during the 9-11 period, post offices, government buildings, and non-governmental facilities were found to be contaminated with anthrax spores. Although numerous individuals were also contaminated, only a few were killed by the associated infection. Severe restrictions were imposed upon USPS mailroom sorting activities.

For many years prior to these activities, and continuing to the present day, the Federal Government together with many other international agencies have devoted significant resources to the prevention and early detection of biological warfare agents. Real-time detection of a bioterrorist attack in progress, of course, depends critically upon the actual agent concentrations present at a vulnerable site. However, lethal concentrations of such agents are often far below the lower limits of real-time detection.

There are other particulates besides bacterial agents that pose significant health threats to an exposed community. These include asbestos fibers, industrially generated particulates such as carbon exhaust constituents, nanofibers, and a variety of byproducts of many manufacturing activities. So-called clean rooms required, for the manufacture of leading edge electromechanical components, are sometimes contaminated with particulates at such low levels that they are rarely detectable.

The most difficult problem associated with attempts to detect such unwanted particles, be they bacteria, bacterial spores, or industrial by-products, is the fact that they are generally at so low a concentration, that their detection signatures (that might easily be measured from a single particle) are very rare events. Thus, for example, monitoring a hospital operating room for the presence of airborne bacteria by flowing a continuous stream of room air through a single particle detection device could never be expected to examine but an extremely small fraction of the total air volume accessible. A screening could only be considered successful if a large fraction of the accessible air could be monitored. Therein lays the basic problem: If one could collect all particles contained within a substantial fraction of the ambient air accessible, one would be faced with the additional problem of distinguishing the particles of importance (e. g. bacteria in a hospital patient care environment) from the probable large quantities of unimportant particles.

Shortly after the Department of Homeland Security (DHS) was formed, it acquired many of the facilities of the Environmental Protection Agency. One of particular importance was the Agency's aerosol particle collection facilities. Such facilities collected a wide range of ambient aerosol particulates that had been captured on large filters. Periodically, these filters were retrieved and their particulate loads examined for bacteria and other signs of biological agent activity. To date, very little bacterial activity was found within the relatively large particulate presence captured/examined.

Bioaerosol sampling methods represent a broad area of active and on-going development, primarily directed to the early detection and identification of the bacterial content of room air. Although specific fractions of typical room air are often deposited onto plates of bacterial growth media and subsequently incubated to confirm the growth thereon of airborne bacteria, the times required to detect such growth are often many hours within which time the remaining airborne bacteria will continue to pose a threat. Additionally, if such organisms are dead and do not grow, their presence while viable cannot be deduced. In addition to the direct attempts to capture and confirm airborne bacterial cells, there are a variety of active sampling devices such as impingers, cyclones, and impactors used to collect all particles present within large volumes of ambient air within small volumes of liquids such as water. A summary of all such processes and liquid borne collection methods are presented and discussed in an article by C. V. Haig et al. in the Journal of Hospital Infection, volume 93, pages 242 to 255 (2016). Although such collection means are expected to be rich in particulates, the means by which, for example, the bacterial fractions therein may be isolated and identified rapidly are not available.

The focus of this patent is an apparatus and method to permit the early examination of the particulate content captured within such aforementioned liquids and to classify and extract therefrom the very small, specifically targeted contents sought.

### BRIEF DESCRIPTION OF THE DRAWINGS

FIG. 1 shows the structure of the preferred A4F embodiment of the means to fractionate airborne particles collected and captured in a liquid medium.

FIG. 2 shows the flow structure produced by the hollow fiber implementation of the field flow fractionation process.

FIG. 3 shows the light scattered at 90° from an eluting mixture of five different polystyrene latex particles fractionated by A4F means.

FIG. 4 shows the light scattered at 90° from a sample of 500 nm polystyrene latex particles following A4F fractionation means.

FIG. 5 shows the scattered intensities at all measured angles for the single elution time indicated in FIG. 4.

FIG. 6 shows the scattered light intensity as a function of angle from a suspension of nearly monodisperse Staphylococcus aureus bacteria.

### SUMMARY OF THE INVENTION

This invention is based upon the assumption that a large fraction of the physical volume of a specified closed region may contain at least few particles that may pose a danger to the personnel present in such regions. As mentioned in the BACKGROUND section, such particles may be airborne biological agents such as bacteria and spores introduced intentionally to affect the personnel present, as might occur during a biological attack. They might also be undetected airborne yet viable bacterial cells present in a typical hospital environment, despite significant and on-going efforts to prevent their presence.

The detection, capture, and subsequent classification of these particles begins with the collection of large quantities of all airborne particles within a large volume of the targeted closed region. For a hospital, such regions may be hospital operating rooms, post-surgical facilities, prenatal ward rooms, or even patient recovery rooms. To anticipate a biological attack, most-likely regions may be cafeteria dining areas, corporate reception areas, performing arts auditoriums, shopping malls, etc.

There are already extant devices by which means virtually all the particulates within a large characteristic volume of a potentially targeted region may be collected in a liquid medium of a few milliliters volume. These are described in the referenced article by Haig et al. Since such liquid medium will be expected to contain predominantly particles that are neither of interest nor importance, the need to isolate specifically targeted particles that may be present at extremely low concentrations is addressed directly by the current invention. Following the collection and isolation of such sought particles, they must be presented at a sufficient concentration so that their identification may be determined rapidly. The present invention achieves this objective by means heretofore never previously considered whereby the liquid-borne particles are fractionated by so-called field flow fractionation or similar means, classified during such fractionation by light scattering means, and those fractions containing the targeted particle types, removed for further testing and identification.

### DETAILED DESCRIPTION OF THE INVENTION

Having collected and isolated in a liquid medium airborne particles from relatively large volumes of ambient air by one of the processes referenced in the cited article by Haig et al, the examination, classification, and possible identification of specific classes of particles within the collection becomes the most important and challenging task. This task is challenging because the classes of particles most sought are those whose relative populations are generally the smallest. For example, in a hospital environment, one would expect bacterial content to be among the smallest. Although great emphasis is placed upon removing all such airborne contaminants, it is not unreasonable to expect that their relative populations will be negligible relative to normal dust from floors, clothing, and shoes, etc.

Similarly, for a biological attack within a cafeteria, for example, the ambient air would be expected to be loaded heavily with dust, food particles, cooking oils, condiment traces, etc. An “inserted” bacterial species or even bacterial spores would be expected to have been introduced at vanishingly small, yet potentially lethal concentrations. Thus the requirements for an effective detection and screening strategy may be summarized by actions that:
1) Define the type of airborne particles to be sought;
2) Collect all the particles present within a large volume of air characteristic of that present in a specified spatial region;
3) Fractionate the collected particles by size or other means;
4) Detect and isolate for further study subclasses of particles among the groups collected that correspond to various targeted classes sought; and
5) Increase the populations of targeted size groups when greater concentrations are required for identification and/or further testing.

Before the targeted particles may be detected among the many, and sometimes overwhelming, quantities of particles collected, they must be selectively extracted and isolated from the surrounding plethora of unimportant particles. Therein lies the problem and the power of this invention, viz. some of the specific particles sought have certainly been captured and reside within the sample, yet at such a low concentration that it may not be possible to extract them, let alone confirm their presence and probable identity.

The essential elements of the preferred means by which the airborne particles captured in liquid means may be separated, and targeted particles detected and isolated, is based upon a technique generally called “Field flow fractionation.” Commonly used to derive and measure the distribution of sizes and masses of nanoparticles and molecules in the range from a few nanometers to a few micrometers, the asymmetric flow field flow fractionation embodiment, often referred to simply as A4, is the preferred embodiment of the present invention to separate and classify airborne particles collected and captured in liquid media as discussed above. An important distinction between the particles currently targeted and those measured in the analytical chemistry field flow fractionation technique mentioned is that for the latter application, the samples are known both as to concentration and their expected size range. It is expected that the particles/distributions collected for examination by the present invention would be unknown as to their concentration in the collected sample or their identity.

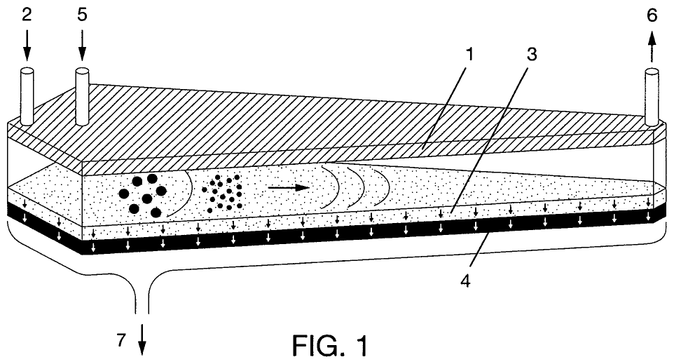

A typical A4F channel is shown in FIG. 1 with an impermeable, generally transparent top plate 1. The channel flow, entering at 2 and flowing between this top plate and a permeable membrane 3 supported on a rigid permeable plate 4, is divided into two parts: one component flows through the permeable membrane 3 with the remainder containing the fractionated sample exiting the channel at 6 where it then passes through suitable detectors, generally based on light scattering. The total flow through the permeable plate 4 is controlled and exits at 7. The sample flow leaving the channel at 6 is just the difference between the inlet flow 2 and exit flow 7. The particle containing sample is injected at 5. By controlling this exit flow 7, the flow perpendicular to the channel is regulated. The injected particles are thus subject to two flow fields as they move through the channel: The flow through the channel from injection at 2 to exit at 6, and the perpendicular flow that leaves through the rigid permeable plate 4 through 7. The channel structure is generally tapered from entrance to exit to compensate somewhat for the net flow per unit area through 7. During the flow down the channel, the particles are separated because of their different sizes and structures. Upon leaving the channel at 6, they will pass through a detection system, generally comprised of a fine laser beam, with the scattered light detected by a surrounding array of detectors, followed by a concentration detection device. The scattered light detectors are most frequently photodiodes. An earlier embodiment of A4F, referred to as cross flow field flow fractionation, provided a flow transverse to the channel by replacing the top impermeable plate with a membrane atop a rigid permeable plate through which a second pump provided the required cross flow.

Other embodiments of the field flow fractionation that might also be applied to fractionate the liquid borne particles based upon their composition include electrical, thermal, and sedimentation implementations. Replacing the cross flow of the A4F implementation, the three other implementations use, respectively, an electric field (produced between two conducting plates), a thermal field (produced between two plates maintained at different temperatures), and a sedimentation field (produced by rotating the flowing sample about an axis perpendicular to the flow direction).

Fractionating the collected liquid-borne particles by thermal and/or electrical flow fractionation means, in addition to A4F, may also be useful in characterizing the various environmental particles present by their associated thermal and electrical response properties. In this manner, any bacterial components present would be further differentiated from the potentially overwhelming background particles.

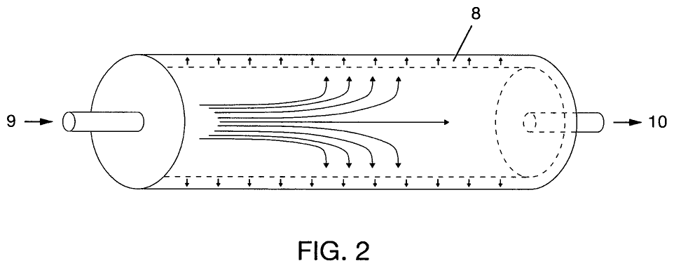

A further simplified, yet very efficient, means for crossflow particle fractionation is the so-called “hollow fiber” device, shown schematically in FIG. 2, based on the use of a porous tube 8 through which a particle-containing fluid 9 is forced to flow against a back pressure provided by a reduced output flow at the end of the tube 10. This excess flow 11 passes radially transverse to the tube length thus providing a separation field similar the cross flow FFF devices. Indeed, because of its simplicity, a hollow fiber device is easily operated using a single syringe pump that may even be hand operated. With appropriate means to provide for light scattering measurement of the eluting sample, the resolution of the device is only somewhat less than the structured A4F device of FIG. 1. Because of its ability to fractionate using easily portable instrumentation components, such a device could well be used to sample liquid collection fluids repeatedly while they are being collected in the field.

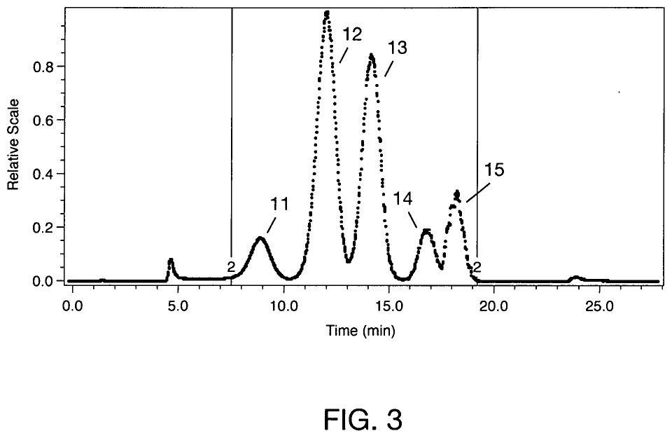

FIG. 3 shows the light scattered as a function of time at 90° following fractionation by A4F means from an eluting mixture of five polystyrene latex spheres of sizes 100, 200, 300, 500, and 1000 nanometers. The smallest 100 nm particles 11 elute first, followed, in time and size by the others 12, 13, 14, 15 of the set. When scattered light measurements are made over a range of angles, the sizes and other structural features of the fractionated/separated eluting particles often may be derived.

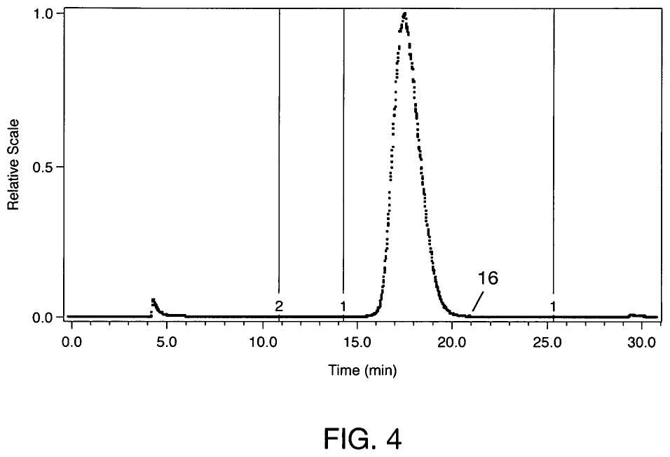

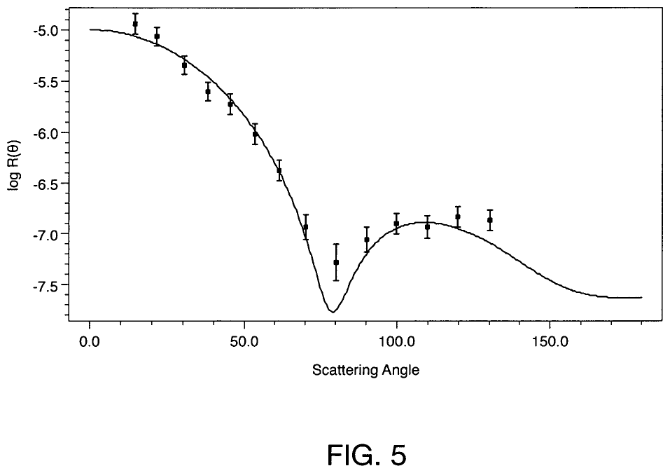

Consider now an elution of fractionated 500 nm particles only. FIG. 4 shows the 90° scattering profile of this sample, as a function of elution time, following A4F fractionation. The vertical indicial mark 16, shown at an elution time of about 21.1 minutes, corresponds to the angular scattering variation shown in FIG. 5. These data, fit to the Lorenz-Mie theory, yield a diameter of 489±8 nm. In water, the total scattering of a single 500 nm polystyrene latex (psi) particle is about equal to the scattering expected from a single S. aureus bacterial cell. Thus, if we could detect and classify a single 500 nm psl particle, we should be able to measure also the scattering in water of a single bacterial cell. The corresponding concentration at the indicia peak shown is less by a factor of 1/3000 of the peak value. For the particular detector array used, the scattering volume detected is about 60 nl=6×10⁻⁵ ml. Thus the scattered light intensities detected by a photo diode array to produce FIG. 5 would be comparable to those scattering from a single 500 nm psi sphere. The laser used for the light scattering measurements shown has an output power of about 130 mW at a wavelength of 658 nm. Naturally, there could be other embodiments of this invention using different wavelengths of incident light as well as different detectors such as photomultipliers instead of the standard photodiodes used to detect the scattering data shown.

Note the scattering minimum that occurs approximately at 80°. The appearance of such minima is characteristic of the scattering of monochromatic light by spherically symmetric particles. As such particle size increases relative to the wavelength of the light incident thereon, this minimum moves toward smaller scattering angles and additional minima appear at larger angles.

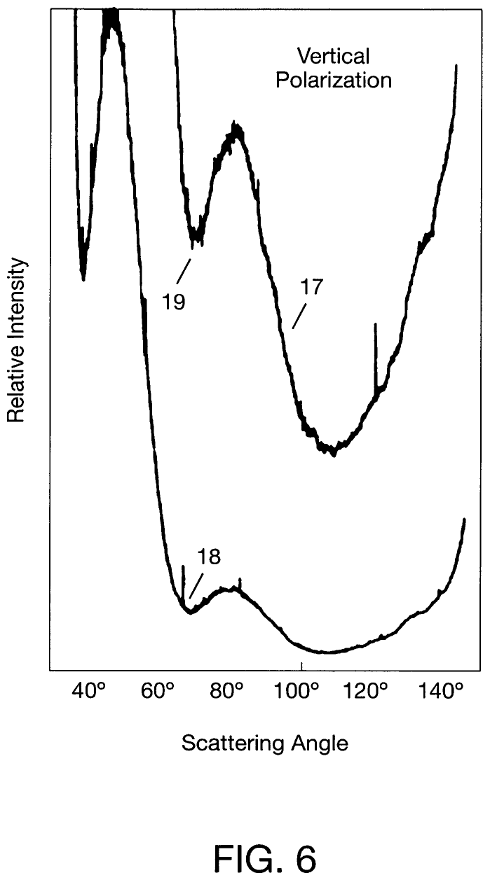

FIG. 6 shows the scattered light intensity (on a linear scale in contrast to the logarithmic scale of FIG. 5) as a function of angle from a suspension in water of nearly monodisperse Staphylococcus aureus bacteria. The higher scattered intensity versus angle curve 17 corresponds to a scalar multiplication of the lower curve by about a factor of 10. From these data, the average cell radius as well as the thickness of their cell wall were derived as shown in the article by Wyatt in pages 277-279 of volume 226 of the journal Nature in 1970. The average radius was derived therefrom to be about 477 nm while the cell wall thickness was 86 nm. Note the corresponding first minimum 18 is at about 65°. The average diameter of these cells is about double that of the 500 nm polystyrene latex spheres of FIG. 5 and this first minimum appears, accordingly, at an angle smaller than the first minimum of the 500 nm particle. Note also the appearance of a secondary minimum occurring at about 110°. The average refractive index of the cells is much smaller than that of the psi particles. In water, the S. aureus bacterial cell and the much smaller psi particle scatter about the same total amount of light.

The inventor hereof has published many articles relating to the so-called inverse scattering problem, i. e. from measurements of the scattered light, a variety of structural features of the scattering particles may be derived. Thus this invention will permit also the classification, and often the identification, of a single scattering particle fractionated from a collection of far more numerous particles of other sizes. The concept represents a powerful tool for the identification/classification of single particles and, therefore, any bacterial cells collected and measured by the invention herein described.

Accordingly, once the airborne particles within a large volume of the targeted region's air content have been captured in a liquid medium, this liquid-borne sample must be searched for the specific targeted classes of particles sought. This is best achieved by the inventive process of fractionating said samples using means such as asymmetric field flow fractionation discussed earlier. For the case of bacteria, for example, these particles will elute after all smaller particles have eluted and been removed by the process. As the smallest particles elute most rapidly, bacteria, bacterial spores, and other larger particles will elute later. It would be expected that bacteria and/or bacterial spore concentrations will be very low relative to common airborne particulates of no interest. If it is necessary to increase the number of suspect particles, additional isolates of the collected fluid-borne aerosols may be processed similarly to provide additional isolates of the suspect sample fraction. However, for the present part of this inventive disclosure, we assume that some targeted particles have been collected and confirmed by measurement of their scattering of light incident upon them. Indeed, the variation of scattered light with angle has long been presented by the inventor as a means for characterization of a variety of particle classes; bacteria among them. Thus particles present in room air collected in a liquid medium would be fractionated by size by such a process disclosing, thereby, samples present at extremely low concentrations. Airborne bacteria and spores captured from the room air and present in the fluid being fractionated would be among such particles. Despite separating possible bacteria from other particulate content captured in the collected room air, the bacterial fraction would be expected to be very small relative to other more frequently present particulates.

Measurements of the light scattered specifically by the isolated fraction may be used therefore to confirm the biological nature of the so-separated bacterial objects. Additionally, such fractions may be isolated during the fractionation process and removed for performing a variety of biochemical tests to supplement and confirm further the classification and even identity of the specific species and spores present in the collected fraction.

Although this disclosure has been focused primarily upon problems relating to the isolation and detection of bacterial residuals within huge concentrations of irrelevant particles, it should be evident to those trained in the art presented that there are many other types of particles whose presence at very low concentrations present additional dangers to persons inhaling them. These include such particles as asbestos as well as the more frequently encountered carbon particles found, for example, in the exhaust of diesel trucks.

1-20. (canceled)

21: A method to search for, isolate, find, and retrieve vanishingly small populations of a specific class of airborne particles that may be present in a selected region of air containing far greater populations of unimportant particles of varying size and composition, comprising the steps of
a. Defining the specific class of airborne particles sought;
b. Collecting all particles present within said selected region of air into a liquid of relatively small volume;
c. Fractionating said collected liquid-borne particles by size or other means;
d. Classifying and/or identifying said fractionated and collected particles from multiangle measurement of light scattered therefrom;
e. Isolating for further study, analysis and/or identification the specific fractions of said collected particles among the types collected whose multiangle light scattering properties correspond to the specific class of particles sought.

22: The method of claim 21 where said specific class of airborne particles sought are bacteria.

23: The method of claim 21 where said specific class of airborne particles sought are bacterial spores.

24: The method of claim 21 where said class of airborne particles sought are asbestos fibers.

25: The method of claim 21 where said region containing particles is in a hospital.

26: The method of claim 21 where said means to fractionate said collected particles is by asymmetric flow field flow fractionation.

27: The method of claim 21 where said means to fractionate said collected particles is by hollow fiber cross flow fractionation.

28: The method of claim 21 where said means to fractionate said collected particles is by electrical field flow fractionation.

29: The method of claim 21 where said means to fractionate said collected particles is by a cross flow field flow fractionation.

30: The method of claim 21 where said means to fractionate said collected particles is by a sedimentation field flow fractionator.

31: The method of claim 21 where said means to collect said particles present is by a liquid sampler.

32: The method of claim 31 where said liquid sampler is an impactor.

33: The method of claim 31 where said liquid sampler is an impinger.

34: The method of claim 31 where said liquid sampler is a cyclonic sampler.

34: The method of claim 21 where such analysis of the specific fractions of said collected particles, among the types collected, that correspond to the defined types of particles sought is achieved by measurement and interpretation of the multiangle scattered light therefrom by each specific fraction.

35: The method of claim 21 where said relatively small volume is a few milliliters.
